(19) United States
(12) Patent Application Publication
(10) Pub. No.: US 2008/0182800 A1
(43) Pub. Date: Jul. 31, 2008

(54) COMPOSITION FOR PREVENTING OR TREATING DEGENERATIVE BRAIN DISEASES COMPRISING A HYDROLYSATE OF GINSENOSIDES

(75) Inventors: In-Hee MOOK, Seongnam-si (KR); Min-Whan Jung, Seongnam-si (KR); Suk-Jae Chung, Seoul (KR)

Correspondence Address: ANDERSON, KILL & OLICK, P.C., 1251 AVENUE OF THE AMERICAS, NEW YORK,, NY, 10020-1182, US

(73) Assignee: DIGITAL BIOTECH CO., LTD., Ansan-si (KR)

(21) Appl. No.: 11/943,988

(22) Filed: Nov. 21, 2007

Related U.S. Application Data
(63) Continuation of application No. 10/514,907, filed on Nov. 16, 2004, now abandoned.

(51) Int. Cl. A61K 31/70; A61P 25/28; A61P 25/16; A61K 31/704; A61K 31/56; A61P 25/00

(52) U.S. Cl. 514/26; 514/23; 514/182

(57) ABSTRACT

A pharmaceutical composition for preventing or treating a degenerative brain disease comprising a compound of formula I or a or a pharmaceutically acceptable salt thereof as an active ingredient: (I) wherein, R¹ is H or Glc-Glc-; R² is H or

[symbol]

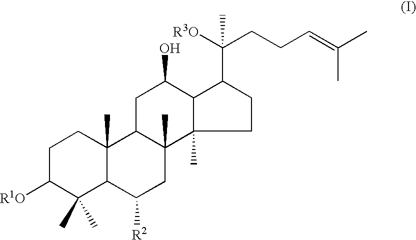

### FIELD OF THE INVENTION

The present invention relates to a composition for preventing or treating a degenerative brain disease comprising a ginsenoside hydrolysate or a pharmaceutically acceptable salt thereof.

### BACKGROUND OF THE INVENTION

Degenerative brain diseases such as senile dementia, Parkinson's disease, cerebral apoplexy and Huntington's disease are mainly caused by the death of nerve cells in the brain.

Senile dementia has become a serious social problem with a sudden increase in older population in the modern society. However, there are no preventive or therapeutic means available yet; hence, this is becoming an economic loss as well. Alzheimer's disease is a major one of the senile dementia, and it has been found that a major cause of the disease is neurotoxicity due to the accumulation of beta-amyloid in the brain (Selkoe, Annu. Rev. Neurosci., 17: 489-517 (1994)). Accordingly, there exists a need for developing a pharmaceutical agent, which blocks the generation or toxicity of beta-amyloid with few side effects.

Parkinson's disease, which is a degenerative disease of the central nervous system (CNS) and frequently occurs in older population, may be accompanied by difficulties in the limb movement and exercising, muscle stiffness and psychological depression. In the brain of a patient suffering from Parkinson's disease, the dopamine level is noticeably lower in the neostriatum due to the death and degeneration of dopaminergic neurons of the substantia nigra (Fahn S., Parkinson's disease in: Diseases of the nervous system, (ED) by A. Asbury, G Mckhann, pp. 1217-1238, Saunders, 1986); hence, the death of dopaminergic neurons is known as the leading cause of the disease.

Known causes of the neuronal death accompanying Parkinson's disease include oxidative stress, metabolic disorder, mutation of mitochondria genes, excitatory amino acid toxicity, and change in the calcium concentration (Fahn, S. and Cohen, G., Ann. Neurol., 32(6): 804-812 (1992); Foley P. and Riederer P., J. Neurol., 247-[Sppl.2] II/82-II/94 (2000)).

Cerebral apoplexy is one of the most common brain diseases and is caused by the death of neurons due to the lack of oxygen or energy resulting from a sudden angiostenosis or hemorrhage. Major causes of the death of neurons in cerebral apoplexy are 1) glutamate exitotoxicity; 2) oxidative toxicity (oxidative stress or free radical toxicity); and 3) apoptosis (or programmed cell death).

Huntington's disease is one of the genetic diseases in the nervous system. It is caused by the loss of neurons in the basal ganglia and cerebral cortex, and oxidative stress is known to play an important role in the death of neurons (Gutekunst C. A., Norflus F., and Hersch S. M., Curr. Opin. Neural, 13:445-450 (2000)).

Oxidative stress due to free radicals is reported to be the leading mechanism of cell death in neurological diseases (Schapira, A. H., Curr. Opin. Neurol., 9(4): 260-264 (1996)), as is evidenced by: increased production of reactive oxygen species after ischemia and suppression of ischemic neuronal death by an antioxidant (Chan, P. H., J. Neurotrauma., 9 Suppl 2:S417-23 (1992)); the production of free radicals through oxidation of dopamine in the substantia nigra of the brain of a Parkinson's patient (Sofic, E. et al., J. Neural Transm., 74:199-205 (1988); Fahn & Cohen, supra); an increase in Fe²⁺ in the corpus striatum of a Huntington's patient (Dexter, D. T. et al, Ann Neuroal, 32 Suppl: 894-100 (1992)); and the generation of free radicals by beta-amyloid in Alzheimer's disease (Richardson, J. S., Zhou, Y., and Kumar, U., Ann N.Y. Acad. Sci., 777:362-367 (1996)), etc. Accordingly, suppression of the neuronal death induced by oxidative stress is an important target in developing a treating agent for degenerative neurological diseases.

Further, a substance that can cross the blood brain barrier (BBB) to promote synapse formation between the existing neurons, thereby regenerating the nervous system, is desired as a treating agent for degenerative neurological diseases; however, such substance has not yet been reported.

The present inventors have reported that ginsenosides Rb1 and Rg1 isolated from Panax ginseng can protect neurons from the toxicity of beta-amyloid (Korean Patent Publication No. 2000-6625). Ginseng is expected to have no toxicity or cause few side effects since it has been used for thousands of years as a herbal medicine.

The present inventors have endeavored to find a substance having a low molecular weight that can cross the BBB and suppress the toxicity of beta-amyloid. Consequently, the present inventors discovered that hydrolysates of ginsenosides, i.e., compounds Y and K, ginsenoside Mc, protopanaxadiol (PPD), and protopanaxatriol (PPT), and ginsenoside Rc can block generation and toxicity of beta-amyloid and protect neurons.

### SUMMARY OF THE INVENTION

Accordingly, it is a major objective of the present invention to provide a pharmaceutical composition comprising an active ingredient for preventing and treating a degenerative brain disease.

In accordance with one aspect of the present invention, there is provided a composition for preventing and treating a degenerative brain disease comprising the compound of formula (I) or a pharmaceutically acceptable salt thereof as an active ingredient:

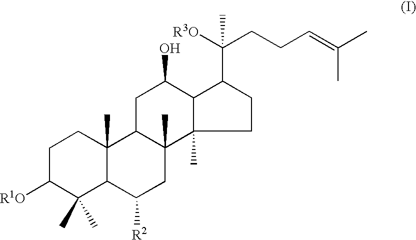

wherein,

### R¹ is H or Glc-Glc-;

### R² is H or OH;

R³ is H, glucose, Ara(p)-Glc- or Ara(f)-Glc-;

### Glc is Glucose;

Ara(p) is arabinose in pyranose form; and
Ara(f) is arabinose in furanose form.

### BRIEF DESCRIPTION OF THE DRAWINGS

The above and other objects and features of the present invention will become apparent from the following description of the invention, when taken in conjunction with the accompanying drawings, which respectively show:

FIG. 1: the result of MTT test showing the effects of ginsenosides Rc and Mc, compound X, PPD and PPT for blocking the toxicity of beta-amyloid.

FIG. 2: the result of MTT test showing the concentration-dependent effect of PPT for blocking beta-amyloid toxicity.

FIG. 3: the result of MTT test showing the effects of compound N, PPD and PPT for blocking the toxicity of beta-amyloid through their anti-oxidative activity.

FIG. 4: the result of MTT test showing the effects of ginsenosides Rc and Mc, compound Y, PPD and PPT on apoptosis induced by staurosporin.

FIG. 5: the result of Western blotting confirming the activity of PPD and PPT for increasing the secretion of α-secretase that cleaves beta-amyloid.

FIG. 6: the result of a Morris water maze test showing the improvement of spatial learning of mice by the administration of ginsenosides Rc and Mc, or compound Y.

FIGS. 7a and 7b: the results of Western blotting confirming the activity of PPD and PPT for increasing the amount of synaptophysin.

FIGS. 5a and 8b: LC-mass peaks of ginsenosides Rg1 and Rb1, respectively, in a brain tissue sample of a rat administered with ginsenosides Rg1 or Rb1.

FIGS. 9a and 9b: LC-mass scans for quantifying ginsenosides Rg1 and Rb1 in a plasma sample.

FIGS. 10a and 10b: LC-mass peaks of PPT and PPD, respectively, in a brain tissue sample of a rat administered with ginsenosides Rg1 or Rb1.

FIGS. 11a and 11b: time-dependent changes in the concentrations of ginsenosides Rg1 and Rb1, respectively, in the plasma and brain samples of a rat administered with ginsenosides Rg1 or Rb1.

FIGS. 12a and 12b: time-dependent changes in the concentrations of PPT and PPD, respectively, in the plasma and brain samples of a rat administered with ginsenosides Rg1 or Rb1.

### DETAILED DESCRIPTION OF THE INVENTION

Among the compounds of formula (I) for use in the inventive pharmaceutical composition for preventing and treating a degenerative brain disease, preferred are those shown in Table 1.

TABLE 1

| Compound | R¹ | R² | R³ |
|---|---|---|---|
| PPD | H | H | H |
| PPT | H | OH | H |
| Compound Y | H | H | Ara(p)-Glc- |
| Compound K | H | H | Glc |
| Ginsenoside Mc | H | H | Ara(f)-Glc- |
| Ginsenoside Rc | Glc-Glc- | H | Ara(f)-Glc- |

Most preferable compounds of formula (I) for the inventive composition are protopanaxadiol (PPD) and protopanaxatriol (PPT).

The compounds of formula (I) can be prepared into various pharmaceutically acceptable salts, for instance, inorganic metal salts such as alkali metal salts (e.g., sodium salt and calcium salt) and alkali earth metal salts (e.g., magnesium salt and calcium salt), ammonium salts, and organic basic salts (e.g., trimethylamine salt, triethylamine salt, pyridine salt, and picoline salt), in accordance with a conventional method.

The compounds of formula (I) prevent the toxicity and generation of beta-amyloid, protect neurons through anti-oxidative activity, permeate through the BBB in vivo to improve spatial learning and increase synaptic density and, thereby, exhibiting excellent preventing and treating effects on the degenerative brain diseases such as senile dementia, Parkinson's disease, cerebral apoplexy and Huntington's disease. Further, the inventive compounds inhibit the generation of beta-amyloid either by enhancing the activity of α-secretase, which cleaves beta-amyloid, or by suppressing β-secretase activity essential for the production of beta-amyloid.

A pharmaceutical formulation may be prepared in accordance with any of the conventional procedures. In preparing the formulation, the active ingredient is preferably admixed or diluted with a carrier, or enclosed within a carrier, which may be in the form of a capsule, sachet or other container. Thus, the formulations may be in the form of a tablet, pill, powder, sachet, elixir, suspension, emulsion, solution, syrup, aerosol, soft and hard gelatin capsule, sterile injectable solution, sterile packaged powder and the like.

Examples of suitable carriers, excipients, and diluents are lactose, dextrose, sucrose, sorbitol, mannitol, calcium silicate, cellulose, methylcellulose, microcrystalline cellulose, polyvinylpyrrolidone, water, methylhydroxybenzoates, propylhydroxybenzoates, talc, magnesium stearate and mineral oil. The formulations may additionally include fillers, anti-agglutinating agents, lubricating agents, wetting agents, flavoring agents, emulsifiers, preservatives and the like. The compositions of the invention may be formulated so as to provide quick, sustained or delayed release of the active ingredient after their administration to a mammal by employing any of the procedures well known in the art.

The pharmaceutical composition of the present invention can be administered via various routes including oral, transdermal, subcutaneous, intravenous and intramuscular introduction. In case of human, a typical daily dose of the composition may range from about 1 to 100 mg/kg body weight, preferably 1 to 10 mg/kg body weight, and can be administered in a single dose or in divided doses. However, it should be understood that the amount of the active ingredient actually administered ought to be determined in light of various relevant factors including the condition to be treated, the chosen route of administration, the age, sex and body weight of the individual patient, and the severity of the patient's symptom; and, therefore, the above dose should not be intended to limit the scope of the invention in any way.

The following Examples are intended to further illustrate the present invention without limiting its scope.

### REFERENCE EXAMPLE

### Test compound

Compounds Y and K, ginsenosides Mc and Rc, protopanaxadiol (PPD) and protopanaxatriol (PPT) used in the examples were prepared in accordance with the method disclosed in the literature (Advances in Ginseng Research, 1998, The Korean Society of Ginseng).

### Example 1

### Effect of Preventing the Toxicity of Beta-Amyloid

In order to examine the activities of the compounds prepared in the Reference Example in preventing the toxicity of beta-amyloid, the MTT analysis (Gillardon, F. et al., Brain Research, No. 6: pp. 169-172 (1996)) was carried out as follows.

5×10³ cells of neuroblastoma B103 cell line (donated by Dr. David Schubert of the Salk Institute, USA) were placed in each well of a 96-well plate with 100 μl of DMEM (Dulbecco's Minimal Eagle's Medium) containing 10% fetal bovine serum(FBS), and cultured at 37° C. under 8% CO₂ for 1 day. After changing the medium to serum-free DMEM, the cells were treated with various concentrations of test compounds and, 1 hour later, with 25 μM of beta-amyloid (25-35 peptide; US peptide, CA, USA). The cells were cultured at 37° C. for 18 hours, treated with 15 μl of MTT solution [10 mg/ml solution of 3-(4,5-dimethyl-2-hiazolyl)-2,5-diphenyl-2H-tetrazolium bromide in phosphate buffered saline (PBS)], and allowed to react at 37° C. for 3 to 4 hours. 100 μl of lysis buffer (10% SDS, 50% dimethylformamide, pH 4.7) was added to the wells, and the cells were cultured overnight at room temperature and relative humidity of 90%. Optical density (OD) of each well was measured at 570 nm and 630 nm, respectively, using a scanning multiwell spectrophotometer, and then the OD value at 630 nm was subtracted from that at 570 nm.

Setting the survival rate of the control group treated only with beta-amyloid as 100%, relative survival rates of the experimental groups treated with compound N; ginsenosides Mc and Rc, PPD and PPT, respectively, were calculated, and the results are shown in Table 2 and FIG. 1. The obtained values were statistically significant.

TABLE 2

| | Concentration of test | |
|---|---|---|
| Test Compound | compound (μM) | Survival rate (%) |
| Beta-amyloid (Aβ) | 25 | 100 |
| Rc | 1 | 161.0 ± 4.9 |
| Rc + Aβ | 1 + 25 | 106.0 ± 0.9 |
| Rc | 10 | 126.7 ± 3.7 |
| Rc + Aβ | 10 + 25 | 107.2 ± 1.9 |
| Y | 1 | 157.7 ± 3.2 |
| Y + Aβ | 1 + 25 | 102.8 ± 1.5 |
| Y | 10 | 123.5 ± 4.6 |
| Y + Aβ | 10 + 25 | 115.5 ± 3.5 |
| Mc | 1 | 148.0 ± 6.3 |
| Mc + Aβ | 1 + 25 | 106.1 ± 2.4 |
| PPD | 1 | 137.7 ± 4.8 |
| PPD + Aβ | 1 + 25 | 106.7 ± 6.9 |
| PPT | 1 | 157.4 ± 3.3 |
| PPT + Aβ | 1 + 25 | 106.6 ± 1.2 |
| PPT | 10 | 140.3 ± 6.4 |
| PPT + Aβ | 10 + 25 | 119.7 ± 8.5 |

When the same procedure as above was repeated except that the cells were treated simultaneously with the test compound and beta-amyloid, or treated with the test compound one hour after the beta-amyloid treatment, the results were almost the same as above. In all cases, the results confirmed that all of the test compounds prevented the beta-amyloid toxicity.

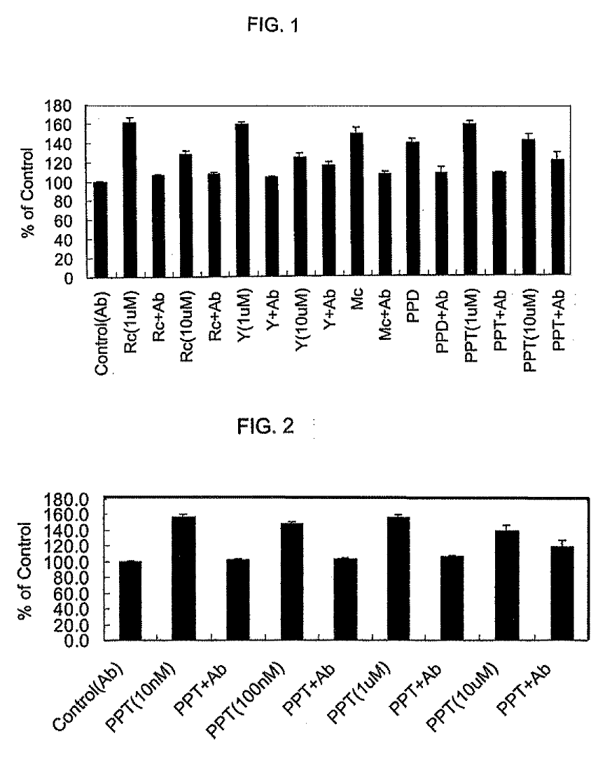

Further, in order to assess the concentration-dependent activity of a test compound, the above procedure was repeated using PPT at concentrations ranging from 10 nM to 10 μM. As can be seen from FIG. 2, PPT began to show its inhibitory activity at the μM level.

### Example 2

### Anti-Oxidative Effect

The prevention of beta-amyloid toxicity has been proposed to be attributable to the blockage of oxidative stress (or damage of cells by free radicals) or the blockage of apoptosis (Becl, C. et al., Cell, 77: pp 817-827 (1994); Yamatsuji, T. et al, Science, 272: pp 1349-1352 (1996)).

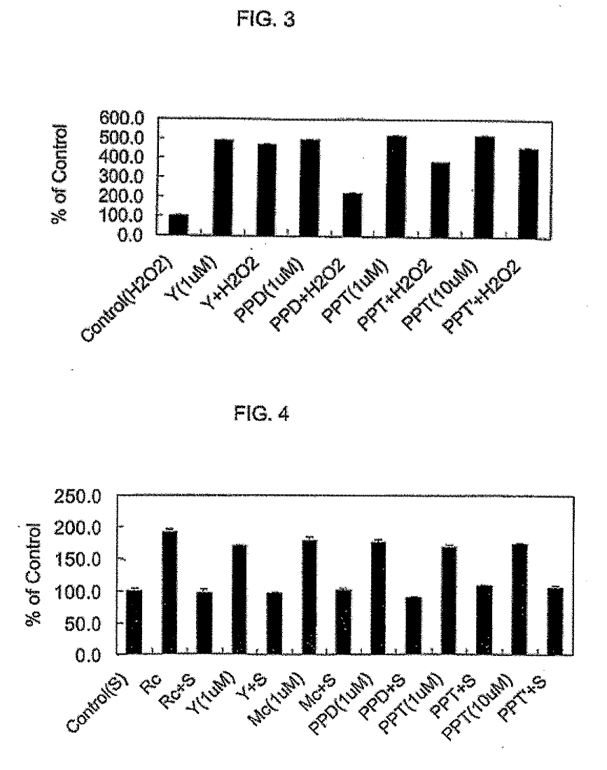

In order to examine whether compounds originating from ginseng can block oxidative stress, the procedure of Example 1 was repeated except for using 1 μM of H₂O₂ in place of beta-amyloid, and the survival rates of the neural cells were determined. The results in Table 3 and FIG. 3 show that the test compounds prevented neuronal death caused by H₂O₂ and, accordingly, can act as anti-oxidative agents.

TABLE 3

| | Concentration of test | |
|---|---|---|
| Test Compound | compound (μM) | Survival rate (%) |
| H₂O₂ | 1 | 100 |
| Y | 1 | 486.5 ± 2.1 |
| Y + H₂O₂ | 1 + 1 | 469.1 ± 5.2 |
| PPD | 1 | 494.3 ± 3.5 |
| PPD + H₂O₂ | 1 + 1 | 219.6 ± 3.0 |
| PPT | 1 | 516.1 ± 1.6 |
| PPT + H₂O₂ | 1 + 1 | 381.3 ± 0.9 |
| PPT | 10 | 516.5 ± 1.2 |
| PPT + H₂O₂ | 10 + 1 | 457.8 ± 2.5 |

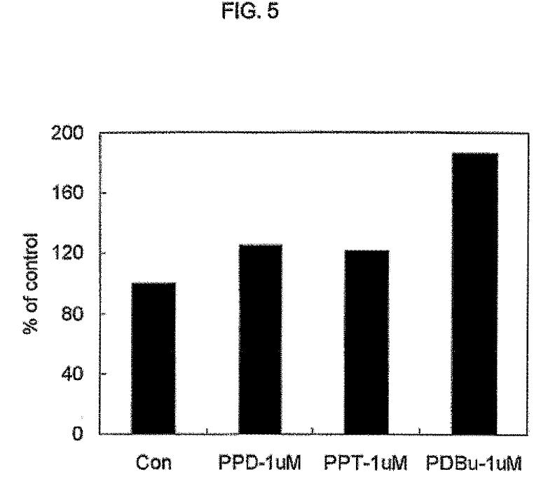

Further, in order to examine whether compounds originating from ginseng can block apoptosis, the procedure of Example 1 was repeated except for using 0.1 μM of staurosporin in place of beta-amyloid to induce apoptosis, and the survival rates of the neural cells were determined. The results in Table 4 and FIG. 4 show that the test compounds did not block apoptosis.

TABLE 4

| | Concentration of test | |
|---|---|---|
| Test Compound | compound (μM) | Survival rate (%) |
| Staurosporin (S) | 0.1 | 100 |
| Rc | 1 | 191.4 ± 4.9 |
| Rc + S | 1 + 0.1 | 97.0 ± 4.8 |
| Y | 1 | 170.3 ± 2.1 |
| Y + S | 1 + 0.1 | 96.7 ± 2.0 |
| Mc | 1 | 178.6 ± 6.3 |
| Mc + S | 1 + 0.1 | 101.1 ± 2.7 |
| PPD | 1 | 176.5 ± 3.5 |
| PPD + S | 1 + 0.1 | 89.2 ± 1.0 |
| PPT | 1 | 169.7 ± 1.6 |
| PPT + S | 1 + 0.1 | 107.8 ± 2.3 |
| PPT | 10 | 173.1 ± 1.2 |
| PPT + S | 10 + 0.1 | 104.6 ± 2.7 |

The above results demonstrate that the test compounds prevent beta-amyloid toxicity by blocking oxidative stress rather then apoptosis.

### Example 3

### Increase in Secretion of sAPP α (α-Secretase Derived Secreted form of Amyloid Precursor Protein)

In order to examine whether the ginseng-originated compounds can inhibit beta-amyloid production, the activity of α-secretase, which cleaves beta-amyloid, was examined.

1×10⁶ cells of neuroblastoma B103 cell line (donated by Dr. David Schubert of the Salk Institute, USA), which secretes APP (amyloid precursor protein), were plated on a 100 mm dish with 100 μl of DMEM medium containing 10% FBS (Fetal Bovine Serum) and cultured overnight at 37° C. under 8% CO₂. The medium was replaced with serum-free DMEM (Gibco, USA), and PPD, PPT or PDBu (phorbol-12, 13-dibutyrate: Sigma Co., USA) was added thereto at a concentration of 1 μM each. The cells were cultured further for 18 hours. The supernatant was isolated from each culture solution and concentrated through a concentration filter (viva spin centricon, obtained from: Vivascience, Binbrook Hill, Binbrook, England). The concentrate was prepared to have a protein concentration of 2 μg/ml and then subjected to electrophoresis on a 8% SDS-PAGE gel.

Protein bands on the gel were transferred to PVDF (polyvinylidene fluoride) membrane (Millipore Co., Bedford, Mass., USA) and blocked with 5% skim milk. The bands on PVDF were reacted with sAPP α-specific antibody 6E10 (1:2000 dilution, Cat. No. 320-05, Senetek, Maryland heights, Mo., USA) at room temperature for 2 hours. Thereafter, the membrane was washed three times with PBS and reacted with 1:3000 dilution of the secondary antibody, anti-mouse IgG (Amersham Pharmacia, Piscataway, N.J., USA) at room temperature for 1 hour. ECL (enhanced chemiluminescence) detection kit (Amersham Pharmacia, Piscataway, N.J., USA) was used to confirm protein bands on the membrane. The protein bands were then quantified via image analysis.

Consequently, sAPP a secretion level was higher in the experimental groups treated with PPD or PPT, compared with that of the untreated control (FIG. 5).

### Example 4

### β-Secretase Inhibitory Activity

The following experiments were carried out in order to test whether the ginseng-originated compounds inhibit the activity of beta-secretase which is essential for beta-amyloid production. Peptide H4848 (Bachem), a known suppressor of beta-secretase, was used for a comparative agent and PPT was used as a test compound.

1.5×10⁶ cells of neuroblastoma B103 cell line secreting APP were plated on a 100 mm dish with DMEM medium containing 10% FBS, and cultured overnight. Then, the cells were lysed up with MEST buffer (20 mM MES, 0.15 M NaCl, 0.5% Triton X-100, pH 6.0 (Bachem)) to obtain a cell extract.

200 mM sodium acetate buffer (ph 4.5), distilled water, beta-secretase inhibitor, 5 μM M2465 (Bachem) as a substrate for beta-secretase and MEST buffer were mixed in order, in amounts as shown in Table 5. The mixtures were reacted for 10 minutes at 37° C. and the cell extract was added thereto in the amount shown in Table 5.

TABLE 5

| | | Amount added (μl) | | | | | |
|---|---|---|---|---|---|---|---|
| | | | | Enzyme | | MEST | Cell |
| | | Buffer | D.W. | inhibitor | Substrate | buffer | extract |
| Blank | | 5 | 37.5 | . | 2.5 | 5 | . |
| Control | | 5 | 37.5 | . | 2.5 | 3.8 | 1.2 |
| Comparative group | | 5 | 32.5 | 5 | 2.5 | 3.8 | 1.2 |
| H4848 (100 nM) | | | | | | | |
| Test | PPT | 5 | 32.5 | 5 | 2.5 | 3.8 | 1.2 |
| groups | (100 nM) | | | | | | |
| | PPT | 5 | 32.5 | 5 | 2.5 | 3.8 | 1.2 |
| | (1 μM) | | | | | | |
| | PPT | 5 | 32.5 | 5 | 2.5 | 3.8 | 1.2 |
| | (10 μM) | | | | | | |
| | PPT | 5 | 32.5 | 5 | 2.5 | 3.8 | 1.2 |
| | (100 μM) | | | | | | |

The resulting mixtures were reacted at 37° C. for 2 hours, and then read by a fluorometer with 360 nm excitation and 508 nm emission wavelengths. Enzyme inhibitory activity was determined based on the enzyme activity of the control.

As a result, the test groups exhibited beta-secretase inhibitory activities as good as the comparative group and, as shown in Table 6, the inhibitory activity increased as the concentration of PPT increased.

TABLE 6

Concentration of PPT

| | 100 nM | 1 μM | 10 μM | 100 μM |
|---|---|---|---|---|
| Enzyme Inhibition | 0 | 0.6 | 3.31 | 18.1 |
| (% of control) | | | | |

### Example 5

### In Vivo Behavioral Test

In order to examine the in vivo activities of ginsenosides Rc and Mc, compounds Y and K, PPD and PPT, which exhibited in vitro preventive effect on beta-amyloid toxicity, a Morris water maze test was carried out in accordance with the method of Mook-Jung et al. (J. Neuro. Res., 63: pp 509-515 (2001)).

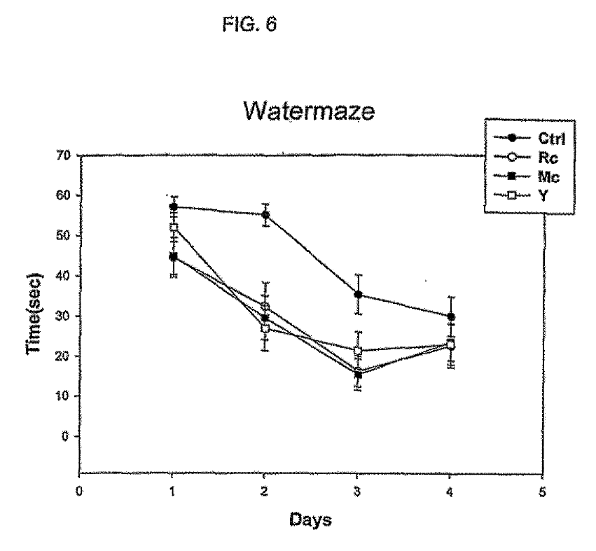

As a result, as can be seen in FIG. 6, the mice of the ginsenoside Rc or Mc-, or compound Y-administered group took much shorter time to reach the platform than the mice of the control group. Also, similar results were shown for the mice of compound K, PPD or PPT-administered group.

These results mean that intellectual functions, especially spatial learning, can be improved by administering ginseng-originated ginsenosides Rc and Mc, compounds Y and K, PPD, and PPT.

### Example 6

### Increase in Synaptic Density

1 mg/kg of ginsenoside Rc or Mc, or compound Y or K, or 0.5 mg/kg of PPD or PPT was intraperitoneally administered to C57BL/6 mice (9-week old, weight: about 25 g) once a day for 4 days. Then, the brain of the mouse was removed and the hippocampus and cortex were separated therefrom. The amounts of synaptophysin, a protein marker of synaptic vesicle, in the hippocampus and cortex were quantified with Western blottingand an image analysis with a densitometer (LAS100, Fujiflim, Japan) employing a synaptophysin-specific antibody (Cat #902 314, Boehringer Mannheim, Germany).

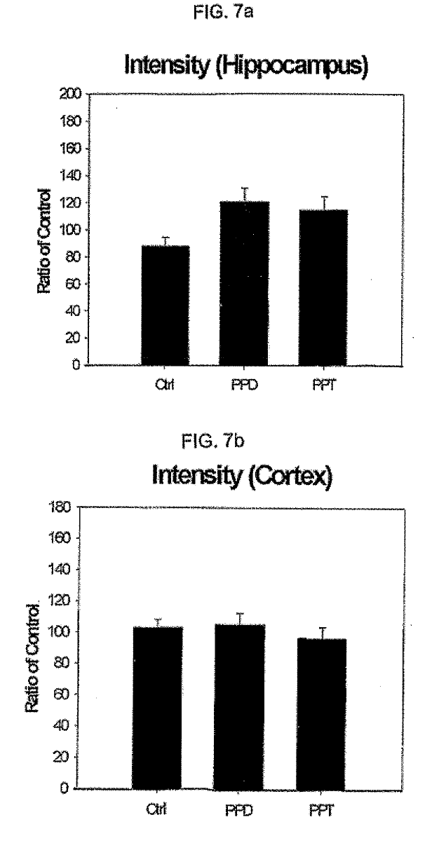

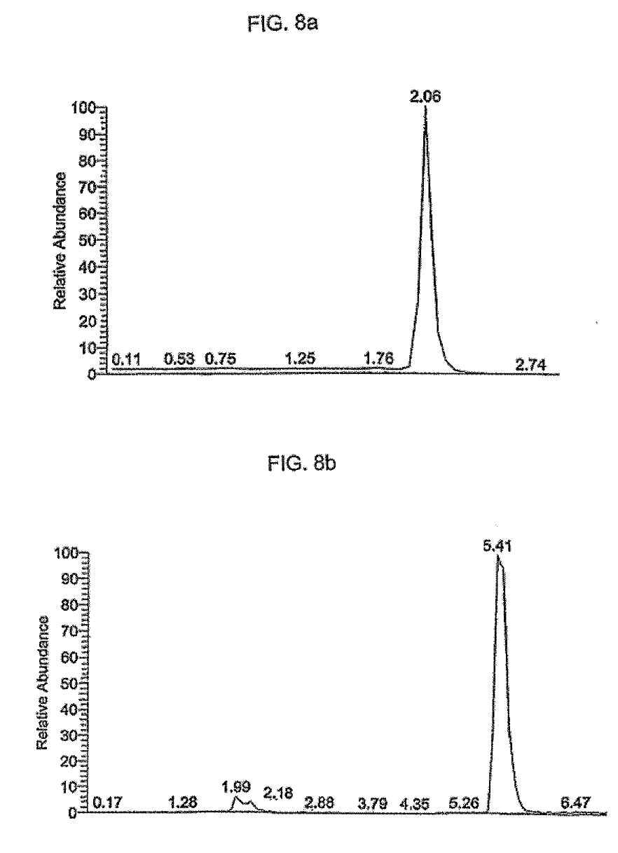

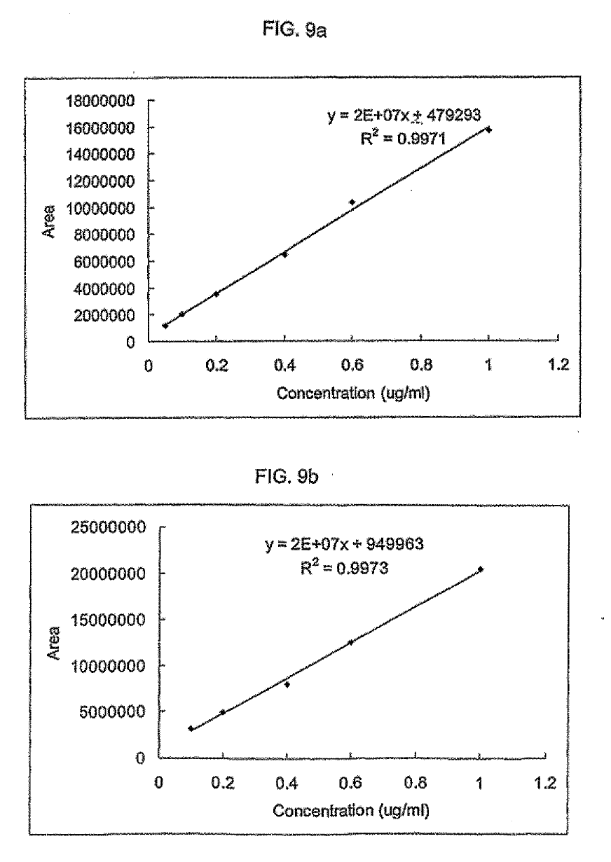

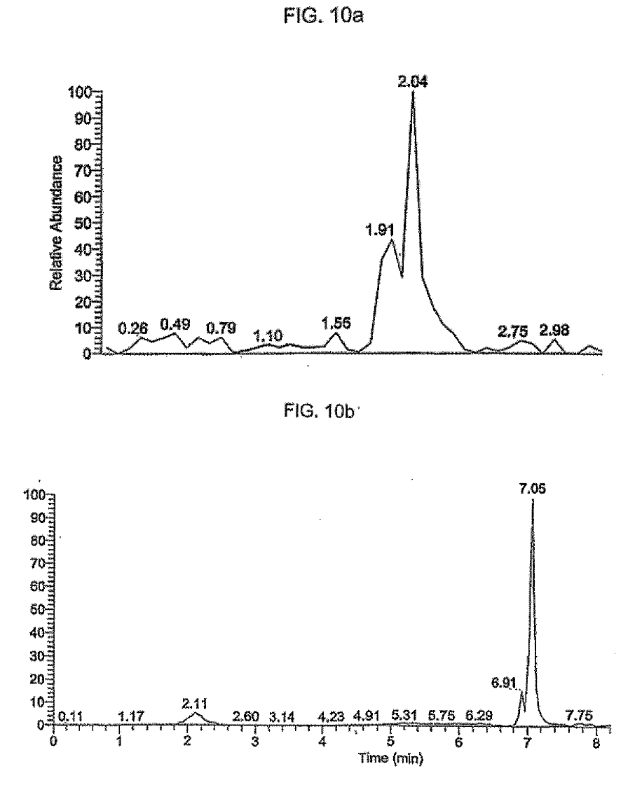

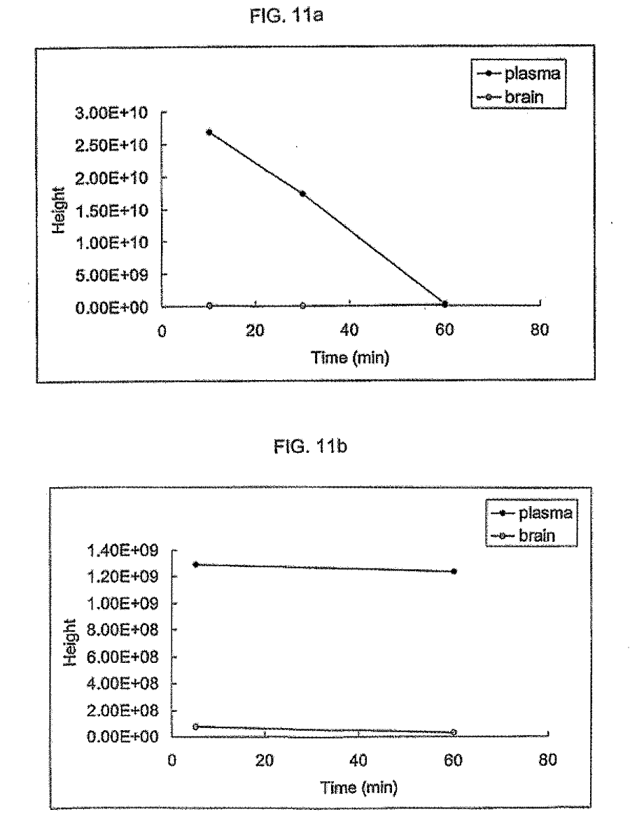

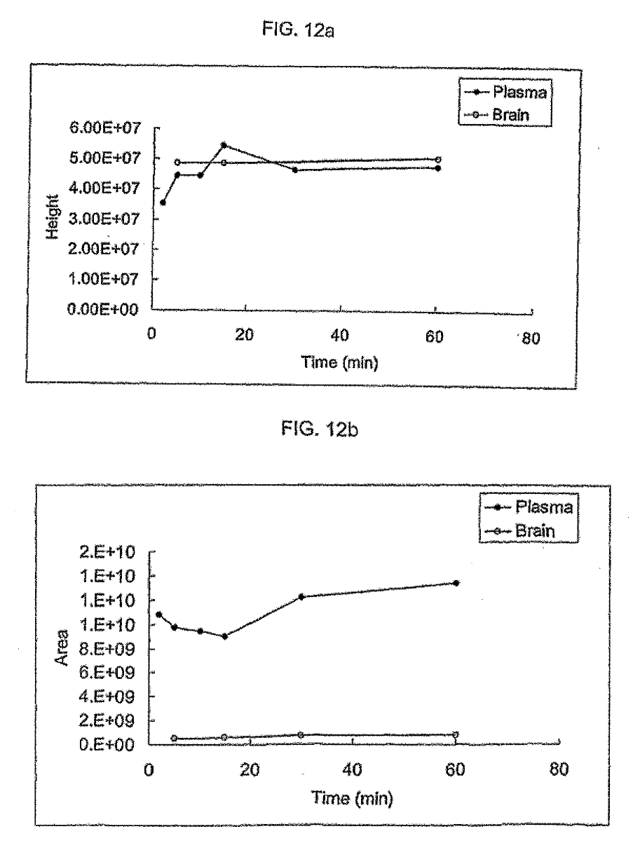

As can be seen in FIGS. 7a and 7b, no change was observed in the mice of the control group, which were administered with vehicle only. However, in the mice of PPD or PPT-administered group, the amount of synaptophysin in hippocampus was significantly increased, while no change was observed in its cortex. This result suggests that intraperitoneally administered PPD and PPT entered into the brain and increased the synaptic density of the neurons in the brain. Also, similar results were observed when the above procedure was repeated with ginsenosides Rc and Mc, and compounds Y and K, respectively.

### Example 7

### Degree of BBB Penetration

In order to determine the active substances that penetrate the BBB and thereby exhibiting the desired activity, upon the administration of ginseng-originated compounds, the following experiments were carried out.

### (Step 1) Preparation of Samples

### (1) Preparation and Quantification of Ginsenoside Samples

1 mg/ml stock solutions of ginsenosides Rg1 and Rb1 (ICN, USA) were serially diluted with acetonitrile to obtain sample solutions having concentrations of 0.1, 0.2, 0.4, 0.6 and 1.0 μg/ml, respectively. 300 μl of each sample solution was placed in a plastic tube and dried in a speed-vac, and then the same amount of acetonitrile was added thereto to obtain 0.3 mt of a standard solution. 20 μl of the standard solution was quantified with a LC mass system [Finnegan LCQ DECA (LC/MSn System)] equipped with a HPLC column (Zorbax Eclipse XDB-C18, 2.1 mm×15 cm) and a mass selective detector. The conditions used for the separation and detection of each compound are as follows:
Quantification of Rg1
Mobile phase-acetonitrile: deionized water (25%:75%)
Flow rate: 0.5 mt/min.
Retention time of the peak: 2.1 min.
Detected ion species: [M+H] (m/z) 801, [M+Na] (m/z) 823 and fragment ion (m/z) 643 were used to identify Rg1, and signals of ESI SIM ms [822.40-825.40] were detected for the quantification of Rg1.
Quantification of Rb1
Mobile phase: gradient of acetonitrile content in a mixture of acetonitrile and deionized water, from 20 to 90% (gradient speed: 20%→90% during 5 min., then 90%→20% during 2 min.)
Flow rate: 0.5 m/min.
Retention time of peak: 5.4 min.
Detected ion species: [M+H] (m/z) 1109, [M+Na] (m/z) 1131 and fragment ion (m/z) 325/365 were used to identify Rb1, and signals of ESI SIM ms [1130.50-1133.50] were detected for the quantification of Rb1.

### (2) Preparation and Quantification of Serum Samples

1 mg/mt stock solutions of ginsenosides Rg1 and Rb1 were serially diluted with acetonitrile to final concentrations of 0.1, 0.2, 0.4, 0.6 and 1.0 μg/ml. 300 μl of the respective solution was placed in a plastic tube and dried in a speed-vac. 100 μl of serum was added thereto and blended with a vortex stirrer. 200 μl of acetonitrile was added thereto and the resulting mixture was blended with a vortex stirrer for 5 min. and centrifuged at 3,000 rpm for 20 min. 270 μl of the resulting supernatant was dried in a speed-vac and 270 μl of acetonitrile was added thereto. 20 μl of the resulting mixture was quantified using the LC mass system described in (1) above.

### (Step 2) Examination of Pharmacokinetics in Blood and Brain

A male Sprague-Dawley rat (weight 250-300 g) was lightly anesthetized with ether and catheterized with a polyethylene tube (PE-50) in the femoral artery and vein. After the rat recovered, a saline solution containing 1 mg/kg of Rg1 or Rb1 was administered into the femoral vein through the intravenous bolus administration. 300 μl blood samples were collected from the rat's femoral artery at 2, 5, 10, 15, 20, 30, 45 and 60 minutes after the administration. Each blood sample was centrifuged to obtain 100 μl of plasma. A plasma sample was prepared as in (2) of step 1 with the plasma. 20 μl of the sample was injected into the LC mass system as in Step 1, and ginsenosides Rg1 and Rb1 and their metabolites PPD and PPT were quantified. The plasma concentrations of ginsenosides and their metabolites were calculated in the forms of weight per plasma (ml) (in case of ginsenosides) or peak size (in case of metabolites).

Immediately after collecting the blood samples, the rats were decapitated and brain tissues were obtained therefrom. The tissues were rinsed with ice-cooled saline, placed in a 15 ml Falcon tube and then weighed precisely. Acetonitrile was added to the tube in an amount twice the weight of the tissue sample, and the sample was homogenized using a tissue homogenizer (Ultra-Turrax T25, JANKE&KUNKEL IKA-Labortechnik). The mixture was centrifuged at 3,000 rpm for 20 minutes. 300 μl of the resulting supernatant was dried in a speed-vac, and 300 μl of acetonitrile was added thereto. 20 μl of the resulting mixture was injected in the LC mass system as in Step 1 to quantify the concentrations of ginsenosides and their metabolites in the brain, and the measured concentrations were represented by weight per gram of the brain tissue or peak size.

Separation and detection conditions for ginsenosides Rg1 and Rb1 were as described in Step 1, and those for metabolites PPD and PPT were as follows:
Quantification of PPT
Mobile phase—acetonitrile: deionized water (25%:75%)
Flow rate: 0.5 ml/min.
Retention time of peak: 2.1 min.
Detected ion species: [M+H] (m/z) 477 and [M+Na] (m/z) 499 were used to identify PPT and signals of ESI SIM mode [497.50-500.50] were detected for the quantification of PPT.
Quantification of PPD
Mobile phase: gradient of acetonitrile content in a mixture of acetonitrile and deionized water, from 20 to 90% (gradient speed: 20%-+90% during 5 min., then 90%→20% during 2 min.)
Flow rate: 0.5 mt/min.
Retention time of peak: 7.1 min.
Detected ion species: [M+H] (m/z) 447 and [M+Na] (m/z) 469 were used to identity PPD, and signals of ESI SIM mode [467.50-470.50] were detected for the quantification of PPD.

### (Step 3) Result and Analysis

### (1) Quantification of Rg1, Rb1, PPD and PPT

The transfer rate of the substances into the brain (K_{brain}) can be represented by the ratio of the mean concentration of the substance in the brain (C_{mean,brain}) to the mean plasma concentration (C_{mean,plasma}) as in Equation 1.

$${K}_{brain} = \frac{{C}_{mean , Brain}}{{C}_{mean , plasma}} \qquad < Equation 1 >$$

The mean concentration of a substance can be determined by dividing the area under the concentration-time curve corresponding to time 0 to t by t and, accordingly, the transfer rate of the substance into the brain (K_{brain}) can be calculated by Equation 2 shown below.

$${K}_{brain} = \frac{\frac{{\int }_{0}^{t} {C}_{brain} \cdot d t}{t}}{\frac{{\int }_{0}^{t} {C}_{plasma} \cdot d t}{t}} = \frac{{AUC}_{brain}}{{AUC}_{plasma}} \qquad < Equation 2 >$$

According to the above LC-mass method, peaks of ginsenosides Rg1 and Rb1 were clearly separated from the endogenous substances in the brain tissue sample obtained from the SD rat administered with 1 mg/kg of Rg1 and Rb1, respectively (FIGS. 5a and 5b). Further, the minimum concentrations of Rg1 and Rb1 detectable with this method from the blood sample obtained from the SD rat were 50 ng/ml and 100 ng/ml, respectively (FIGS. 9a and 9b). Accordingly, this method can be easily applied to the quantification of ginsenosides in a bodily sample.

Further, PPT and PPD peaks were also well detected with the LC-mass method from the brain tissue sample obtained from the SD rat at 15 minutes after the administration of 1 mg/kg of Rg1 or Rb1 thereto (FIGS. 10a and 10b).

### (2) Changes in the Concentrations of Ginsenosides Rg1, Rb1, PPD and PPT in the Blood and Brain

FIGS. 11a and 11b show time-dependent changes in the concentrations of ginsenosides Rg1 and Rb1 in the plasma and brain samples obtained from the SD rats administered with 1 mg/kg of ginsenosides Rg1 or Rb1, respectively. As can be seen from FIGS. 11a and 11b, the concentration of Rg1 in the brain was below the detectable level, and the concentration of Rb1 in the brain was very low until 60 min. as compared with that in the plasma (below 5.8%). Further, when the concentrations of various metabolites of ginsenosides Rg1 and Rb1 in the plasma and brain were examined, the ratio of brain concentration to plasma concentration was below 0.15 for all metabolites except for PPT which is the final metabolite of Rb1. These results demonstrate that ginsenosides Rg1 and Rb1 and their metabolites, except for PPT, do not migrate into the brain. Time-dependent changes in the concentrations of PPT and PPD, respectively, in the plasma and brain samples are shown in FIGS. 12a and 12b, respectively. In the case of PPT, its concentration in the plasma was almost identical to that in the brain tissue. The transfer rate of PPT into the brain calculated with Equations 1 and 2 was 130 ±46.4%.

While the invention has been described with respect to the above specific embodiments, it should be recognized that various modifications and changes may be made to the invention by those skilled in the art which also fall within the scope of the invention as defined by the appended claims.

1-7. (canceled)

8. A method of treating a degenerative brain disease selected from the group consisting of Alzheimer's disease, Parkinson's disease, cerebral apoplexy and Huntington's disease in a mammal, which comprises administering a compound of formula I or a pharmaceutically acceptable salt thereof to the mammal: 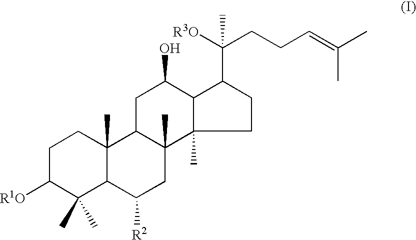
wherein,
R¹ is H or Glc-Glc-;
R² is H or OH;
R⁵ is H, glucose, Ara(p)-Glc- or Ara(F)-Glc-;
Glc is Glucose;
Ara(p) is arabinose in pyranose form; and
Ara(f) is arabinose in furanose form.

9. The method of claim 8, wherein the compound of formula I is selected from the group consisting of ginsenoside Rc, ginsenoside Mc, compound Y, compound K, protopanaxadiol (PPD) and protopanaxatriol (PPT).

10. The method of claim 8, wherein the compound of formula I is protopanaxatriol (PPT).

11. The method of claim 8, wherein the compound of formula I has an anti-oxidative activity.

12. The method of claim 8, wherein the compound of formula I enhances the activity of α-secretase.

13. The method of claim 8, wherein the compound of formula I inhibits the activity of β-secretase.

14. The method of claim 8, wherein the compound of formula I enhances the synaptic density.
